(19) United States
(12) Patent Application Publication
(10) Pub. No.: US 2004/0234628 A1
(43) Pub. Date: Nov. 25, 2004

(54) Topical composition for the treatment of skin disorders and methods of using the same

(76) Inventors: Marcy L. Kearns, Delray Beach, FL (US); Steven R. Strauss, Hillsdale, NJ (US)

Correspondence Address: Kenneth Watov, WATOV & KIPNES, P.C., P.O. Box 247, Princeton Junction, NJ, 08550, US

(21) Appl. No.: 10/851,589

(22) Filed: May 21, 2004

Related U.S. Application Data
(60) Provisional application No. 60/472,492, filed on May 22, 2003.

(51) Int. Cl. A61K 35/78

(52) U.S. Cl. 424/736; 424/745; 424/765; 424/776

(57) ABSTRACT

A topical composition for the treatment of skin disorders such as scars containing at least one, preferably two select botanical oils alone or in combination with at least one other active agent and methods of using the same to treat skin disorders.

### RELATED APPLICATION

The present Application claims priority benefits of U.S. Provisional Patent Application No. 60/472,492 filed May 22, 2003.

### FIELD OF THE INVENTION

The present invention is generally directed to a topical composition for the treatment of skin disorders including the treatment of scar tissues in which one or more of a select group of essential oils is employed in a skin treating effective amount.

### BACKGROUND OF THE INVENTION

Efforts have been made to treat scars and other skin disorders using topical compositions. The advantage of a topical composition is that it can be readily administered by the patient and can be easily administered over a period of time without invasive procedures. Generally, however, topical compositions for the treatment of skin disorders such as scars have not met with great success.

U.S. Pat. No. 5,885,581 discloses a dermatological composition for the treatment of scars containing polyethylene glycol, sorbic acid, allantoin, xanthan gum, and fluid onion extract.

U.S. Pat. No. 5,552,162 describes a method of reducing the size and improving appearance of a scar associated with a fibromatosis, a keloid, or a hypertrophic wound healing disorder in which collagenase activity in the scar is stimulated. The method is carried out by covering the scar with a thermal insulating material that elevates the surface temperature of the scar.

Botanical oils have been used in topical compositions for a variety of purposes. For example, U.S. Pat. No. 5,916,573 discloses a topical grapeseed oil composition which is known to contain powerful antioxidants which can be used to combat the effects of ultraviolet radiation. The composition can contain a hydrating agent which is stated to facilitate cell rehydration including primrose oil or any other botanical essential oil.

U.S. Pat. No. 5,997,889 is directed to a hand and body cream which may be useful for the treatment of eczema which contains almond oil, cocoa butter, jojoba oil, vitamin E oil, beeswax, hydrogenated soybean flakes, and vitamin A oil. The composition may also contain a fragrance which can be selected from a wide variety of fragrances including bergamot, geranium, immortelle, lavender, and the like. These essential oils provide a fragrant aroma to a topical composition containing a variety of active agents.

U.S. Pat. No. 6,361,806 claims an emollient composition containing a mixture of C₁₆ and C₁₈ fatty acids.

There remains a need in the art of treating skin disorders, especially scar tissue, to provide an effective composition that can obtain desirable results in a reasonable period of time.

There is a further need in the art to provide a skin treating composition which can contain a variety of standard skin treating agents in combination with the particular agents used in the composition to obtain the desirable skin treating results.

### SUMMARY OF THE INVENTION

The present invention is generally directed to a topical composition for the treatment of skin disorders especially scar tissue which contains at least one of a select group of botanical oils or essential oils that have been determined to have exceptional skin treating properties alone and in combination with each other. As used herein the terms “botanical oils” and “essential oil” will be used interchangeably and are intended to cover the specific skin treating agents identified hereinafter.

In one aspect of the present invention, there is provided a topical composition for the treatment of skin disorders comprising a skin treating effective amount of at least one skin treating substance selected from the group consisting of Rosa Rubignosa (Rubiginosa) hereafter “Rubignosa” oil, evening primrose oil, immortelle oil, lavender oil, bergamot oil, and geranium bourbon oil, and a suitable topical carrier. In a preferred aspect of the present invention, the skin treating substance is at least one, preferably two botanical oils selected from Rosa Rubignosa oil, evening primrose oil, and immortelle oil. A more preferred composition will contain all three of the preferred botanical oils and a most preferred combination for the aspect of the ability to treat skin disorders, especially scars, will contain all six botanical oils identified above.

The present invention also encompasses methods of using the topical composition to treat various skin disorders, especially scars.

### DETAILED DESCRIPTION OF THE INVENTION

The present invention is directed to a topical composition for the treatment of skin disorders in which a skin treating effective amount of at least one of the select group botanical oils is employed in the composition. The select group of botanical oils includes all forms of Rosa Rubignosa oil, evening primrose oil, immortelle oil, lavender oil, bergamot oil, and geranium bourbon oil. The preferred group of botanical oils includes Rosa Rubignosa oil, evening primrose oil, and immortelle oil wherein at least two of the oils employed in the composition are selected from this group. It will be understood that the select group of botanical oils include all forms thereof. For example, bergamot oil includes Bergamot FCF which is a bergaptine-free oil. In addition, immortelle oil includes the products obtained from Helichrysum Angustifolium and Helichrysum italicum G.D.

Rosa Rubignosa oil, also known as rose hip seed oil, can be obtained from the Rosa Rubignosa plant. This botanical oil carries an exceptionally high concentration of essential fatty acids as well as trans-retinoic acid. Evening primrose oil is obtained from the plant family Oenothera and contains vitamin A and is a source of Omega 3 essential fatty acids. Immortelle oil also known as Helichrysum oil may be extracted from Helichrysum Angustifolium, and Helichrysum italicum G.D. an evergreen aromatic herb.

Of the remaining botanical oils, lavender oil is typically obtained from Lavandula Vera while bergamot is obtained from Citrus Bergamia which is a small tree in the orange tree family. Geranium bourbon is obtained from the plant Pelargonium Graveolens.

The essential oils employed in the present invention are known fragrances for inclusion in topical compositions. However, their combination in skin treating effective amounts produces a topical composition that is particularly effective for the treatment of skin disorders, especially scar tissue.

The select group of essential oils employed in the present invention provide exceptional healing and organoleptic properties (e.g., non-greasy, quick absorbing skin feel and the like) for a topical composition. It is believed that the select group of essential oils, at least in part due to their low molecular weight and small size, are able to interact with the tissues affected by the skin disorder at the cellular level (i.e., the active agent can permeate through the cell membranes). As a consequence, the present composition provides more rapid transdermal delivery of the active agents and more efficient healing of affected tissues than some conventional topical products for treating skin disorders.

The total amount of the skin treating agent employed in the present composition is in the range of from about 0.5 to 10% by weight based on the weight of the topical composition. The amount of the skin treating agent may be selected within this broad range depending on the type of skin disorder and the sensitivity of the patient to the select group of botanical oils. A lower range of from about 0.5 to 1.67% by weight is the most cost effective range for the botanical oils employed in the present invention and is particularly suited for individuals who have sensitive skin or may react to the presence of botanical oils thereon. The typical range for the skin treating agent, especially for patients with low skin sensitivity is in the range from about 1.67% to 6.5% by weight. This range of amounts of the select botanical oils provides potent treatment capability but results in a higher cost for the product and provides an elevated although still relatively low risk of sensitivity reactions.

The most potent range for the skin treating agent of the present composition is from about 6.5 to 10% by weight and is typically reserved for medicinal use on severely injured patients because of the high potency of the botanical oils and due to the elevated potential for sensitivity reactions.

The ranges described above cover the total amount of the skin treating agent within the composition which may be typically apportioned according to the number of the botanical oils present in the composition.

For example, if a relatively low cost topical composition is desired with the highest potency, the range of 0.05% to 1.67% would be selected with the total amount apportioned among the botanical oils appearing in the composition. For example, if two essential oils are employed (e.g., Rosa Rubignosa oil and evening primrose oil), the range of amounts for the lower range concentration for each botanical oil would be from about 0.025% to 1.64% by weight, more typically each of the two essential oils would be present in an amount of from about 0.5 to 1.2% by weight.

The preferred range for a low concentration topical composition in accordance with the present invention employing the three preferred botanical oils is 0.3-0.67% by weight for Rosa Rubignosa, 0.15 to 0.5% by weight of evening primrose oil, and 0.1 to 0.5% by weight of immortelle oil.

The other oils which may be employed in topical compositions of the present invention (i.e., lavender oil, bergamot oil, and geranium bourbon oil) may be present in similar amounts as described above. For example, if all six oils were employed in a mid-range composition having a total effective amount of 1.67% to 6.5% by weight, then each botanical oil could be expected to be within the range of about 0.5% to about 1.6% by weight.

Thus, generally, the amount of each of the botanical oils present in the topical composition of the present invention will fall within the range of 0.05 to 5% by weight. The preferred amounts of the various essential oils will vary slightly (e.g., Rosa Rubignosa oil is from about 0.3 to 0.67% by weight while evening primrose oil is from about 0.15 to 0.5% by weight but will generally be in the range of from about 0.1 to 0.7% by weight).

The topical composition of the present invention may contain a variety of other ingredients necessary to form a suitable composition for a topical application. Typical components of the composition may include vitamin E oil which possesses antibacterial properties and good skin care properties, and is effective as a preservative for the composition. Vitamin E oil may typically be present in an amount of from 0.1 to 10% by weight.

The composition may also contain a first type of carrier oil to provide a suitable matrix for the oil-based ingredients. Typical examples of the first type of carrier oil include jojoba oil, olive oil, peanut oil, and the like. The carrier oil is typically present in amount of from about 5 to 65% by weight.

In addition to the preservative properties provided by vitamin E oil, the composition may contain other preservative to insure suitable shelf life for the product. A typical example of another preservative is Germaben II-E produced by ISP Technologies, Inc., Sutton Laboratories division (Range 0.1%-2.0%) which is a mixture of 60% Propylene glycol, 20% Germaben II, 10% Methyl paraben, and 10% Propyl paraben.

The composition may also contain a surfactant which enables the water and oil combination to be mixed and suitably blended for the purpose of forming a stable emulsion. An example of a suitable surfactant is the polysorbate surfactant (e.g. polysorbate Tween 80) in an amount of from 0.1% to 2% by weight.

A second class of carrier oils that may be used in conjunction with the present composition includes calendula extract or coconut oil in an amount of 1 to 20% by weight. The composition will also typically contain an emulsifier which facilitates absorption of the composition on the skin. Typical examples of the emulsifier include incroquat behenyl TMS (behentrimonium methosulfate and ceteayl alcohol and crodafos ces-ceteayl alcohol in combination with dicetyl phosphate and ceteth-10 phosphate each in amount of from 0.7 to 7% by weight). The balance of the composition will be water in an amount of 10 to 65% by weight.

The composition can be prepared in a variety of topical compositions including creams, ointment, sprays, and lotions, each of which may be prepared in a conventional manner.

The composition of the present invention contains at least one of a select group of botanical oils to provide skin-treating properties for a variety of skin treating conditions including, but not limited to scars, redness and/or inflammation from scars, keloids, burns from any source including heat, sun, or chemicals, damage from ultraviolet rays and other sources of radiation, stretch marks, facial wrinkles, fine lines, and hemorrhoids. The present composition may also accelerate the healing of cuts, wounds, abrasions, blisters, skin irritations, and stop or reduce the intensity of itching from insect bites, stings, and the like.

The topical composition of the present invention employs up to six of a select group of botanical oils. At least one of the oils preferably is a combination of two or more and more preferably all six select essential oils for treatment of a variety of skin disorders. The present composition may also contain other active agents which are selected for the same skin-treating purpose or provide another function. Examples of some such additional active agents include antibiotics, anti-acne medications, analgesics, anti-fungal agents, anti-psoriasis agents, sunscreens, anorectal agents, and anti-wrinkle agents such as retinoids, and the like. These additional active agents may be added to the topical composition of the present invention in conventional amount (e.g., an antibiotic effective amount of an antibiotic agent), typically in an amount of from about 0.1 to 20% by weight.

The incorporation of antibacterial agent provides a topical composition that may be used when a relatively sterile environment is desired. For example, the presence of an antibacterial agent in the present composition will enable the composition to be employed for the treatment of scar tissue arising from surgery shortly after the surgery is performed (e.g. Cesarean section).

The topical composition may be administered as needed, typically at least once a day, more typically 1 to 4 times for day until positive results are obtained typically within a couple of weeks to several months, depending on the type of skin disorder, the severity of the skin disorder, and the concentration of the active ingredients in the composition. The composition is desirably applied evenly over the skin disorder and gently massaged therein for a period of one to two minutes.

The following examples are submitted for illustrative purposes only and are not intended to limit the invention as set forth in the claims forming part of the Application.

### EXAMPLE 1

### Preparation of Low Range Composition Containing All Six Botanical Oils

A cream was prepared containing the ingredients set forth in Table 1 in the following manner.

Jojoba oil, polysorbate Tween 80, natural beeswax, incroquat behenyl TMS and the crodafos ingredient identified in Table 1 were added to a steam jacketed heated vessel equipped with air driven paddle blenders in the relative amounts shown in Table 1 and mixed at a temperature of 55 to 60° until a homogenous oil phase mixture was obtained.

A separate vessel was used to form an aqueous phase. The relative amounts of calendula extract, Germaben II-E, and deionized water were heated to a temperature of from 55 to 60° C. under mixing to obtain a homogenous mixture followed by cooling to 40° C. The oil phase was added to the aqueous phase under mixing to form a combined phase followed by cooling to about 40° C. The botanical oils in the relative amounts set forth in Table 1 were then added to the combined phase under mixing followed by cooling to 33° to 35° C. to produce a cream for topical application.

TABLE 1

| INGREDIENTS | % W/W |
|---|---|
| Rosa Rubignosa Oil | 0.300 |
| Lavender Oil | 0.200 |
| Evening Primrose Oil | 0.150 |
| Bergamot Oil | 0.050 |
| Geranium Bourbon Oil | 0.100 |
| Immortelle Oil | 0.100 |
| Vitamin E Oil (Tocopherol) | 0.250 |
| Jojoba Oil | 45.000 |
| Germaben II-E | 1.000 |
| Polysorbate Tween 80 | 1.000 |
| Natural Beeswax | 4.500 |
| Calendula Extract (Glycerin Base) | 9.000 |
| Incroquat Behenyl TMS—Behentrimonium | 2.700 |
| Methosulfate (and) Ceteayl Alcohol | |
| Crodafos Ces-Ceteayl Alcohol (and) Dicetyl | 2.700 |
| Phosphate (and) Ceteth-10 Phosphate | |
| Deionized Water | 32.950 |
| Total | 100.00 |

### EXAMPLE 2

### Preparation of Mid Range Composition Containing Three Select Essential Oils

A cream is prepared in the same manner as Example 1 containing the ingredients set forth in Table 2.

TABLE 2

| INGREDIENTS | % W/W |
|---|---|
| Rosa Rubignosa Oil | 1.800 |
| Lavender Oil | 1.200 |
| Evening Primrose Oil | 0.800 |
| Vitamin E Oil (Tocopherol) | 0.250 |
| Jojoba Oil | 42.100 |
| Germaben II-E | 1.000 |
| Polysorbate Tween 80 | 1.000 |
| Natural Beeswax | 4.500 |
| Calendula Extract (Glycerin Base) | 9.000 |
| Incroquat Behenyl TMS—Behentrimonium | 2.700 |
| Methosulfate (and) Ceteayl Alcohol | |
| Crodafos Ces-Ceteayl Alcohol (and) Dicetyl | 2.700 |
| Phosphate (and) Ceteth-10 Phosphate | |
| Deionized Water | 32.950 |
| Total | 100.00 |

What is claimed is:

1. A topical composition for the treatment of skin disorders comprising a skin treating effective amount of at least one skin treating substance selected from the group consisting of Rosa Rubignosa (Rubiginosa) Oil, Evening Primrose Oil, Immortelle Oil, Lavender Oil, bergamot oil, and geranium bourbon oil, and a suitable carrier.

2. The topical composition of claim 1 wherein the skin treating substance comprises Rosa Rubignosa Oil and Evening Primrose Oil.

3. The topical composition of claim 1 wherein the skin treating substance comprises Rosa Rubignosa Oil and Immortelle Oil.

4. The topical composition of claim 1 wherein the skin treating substance comprises Evening Primrose Oil and Immortelle Oil.

5. The topical composition of claim 1 wherein the skin treating substance comprises Rosa Rubignosa Oil, Evening Primrose Oil, and Immortelle Oil.

6. The topical composition of claim 1 wherein the skin treating effective amount of the skin treating substance is from about 0.5 to 10% by weight based on the weight of the topical composition.

7. The topical composition of claim 6 wherein the skin treating effective amount of the skin treating substance is from about 0.05 to 1.67% by weight.

8. The topical composition of claim 6 wherein the skin treating effective amount of the skin treating substance is from about 1.67 to 6.5% by weight.

9. The topical composition of claim 6 wherein the skin treating effective amount of the skin treating substance is from about 6.5 to 10% by weight.

10. The topical composition of claim 1 wherein the skin treating effective amount of Rosa Rubignosa Oil is from about 0.05 to 5% by weight.

11. The topical composition of claim 10 wherein the skin treating effective amount of Rosa Rubignosa Oil is from about 0.3 to 0.67% by weight.

12. The topical composition of claim 1 wherein the skin treating effective amount of Evening Primrose Oil is from about 0.05 to 5% by weight.

13. The topical composition of claim 11 wherein the skin treating effective amount of Evening Primrose Oil is from about 0.15 to 0.5% by weight.

14. The topical composition of claim 1 wherein the skin treating effective amount of Immortelle Oil is from about 0.05 to 5% by weight.

15. The topical composition of claim 14 wherein the skin treating effective amount of Immortelle Oil is from about 0.1 to 0.5% by weight.

16. The topical composition of claim 1 wherein the skin treating effective amount of lavender oil is from about 0.05 to 5.0% by weight.

17. The topical composition of claim 16 wherein the skin treating effective amount of lavender oil is from about 0.1 to 0.7% by weight.

18. The topical composition of claim 1 wherein the skin treating effective amount of bergamot oil is from about 0.05 to 5.0% by weight.

19. The topical composition of claim 18 wherein the skin treating effective amount of bergamot oil is from about 0.1 to 0.7% by weight.

20. The topical composition of claim 1 wherein the skin treating effective amount of geranium bourbon oil is from about 0.05 to 5.0% by weight.

21. The topical composition of claim 20 wherein the skin treating effective amount of geranium bourbon oil is from about 0.1 to 0.7% by weight.

22. The topical composition of claim 1 further comprising at least one additive selected from preservatives, emulsifiers, carrier oils, surfactants, and base carriers.

23. The topical composition of claim 1 further comprising at least one active agent selected from the group consisting of antibiotics, anti-acne agents, anti-fungal agents, anti-wrinkle agents, anorectal agents, anti-psoriasis agents, sunscreening agents, and analgesics.

24. The topical composition of claim 22 wherein the amount of the active agent is from about 0.01 to 20% by weight based on the weight of the composition.

25. The topical composition of claim 24 wherein the amount of the active agent is from about 1 to 10%.

26. The topical composition of claim 1 wherein the topical composition is in the form of a cream, ointment, spray, or lotion.

27. The topical composition of claim 1 wherein the skin disorder is selected from the group consisting of scars, redness or inflammation from scars, wounds, abrasions, cuts, blisters, acne, wrinkles, keloids, hemorrhoids, UV damaged skin, stretch marks, wounds, insect bites and stings, itching, burns, and damage from exposure to chemicals, radiation, and combinations thereof.

28. A method of treating a skin disorder comprising topically applying to the affected skin an effective amount of the topical composition of claim 1.

29. The method of claim 28 wherein the topical composition is administered from 1 to 4 times per day.
